(19) United States
(12) Patent Application Publication
(10) Pub. No.: US 2026/0224378 A1
(43) Pub. Date: Aug. 6, 2026

(54) STENT WITH SIDE BRANCH ACCESS

(71) Applicant: BOSTON SCIENTIFIC SCIMED, INC., Maple Grove, MN (US)

(72) Inventors: Robert Sears, Littleton, MA (US); Jonathan Root, Townsend, MA (US); Perry Thomson, Somerville, MA (US); Jessica Rincon Nieto, Boston, MA (US); Fionn Stapleton, Oranmoe (IE); Gary Gilmartin, Foxford (IE); Keith O'Reilly, Kilcolgan (IE); Daniel Tuck, Oughterard (IE)

(73) Assignee: BOSTON SCIENTIFIC SCIMED, INC., Maple Grove, MN (US)

(21) Appl. No.: 19/464,375

(22) Filed: Jan. 29, 2026

Related U.S. Application Data
(60) Provisional application No. 63/753,010, filed on Feb. 3, 2025.

(51) Int. Cl. A61F 2/89

(52) U.S. Cl. CPC A61F 2/89; A61F 2220/0016; A61F 2230/0069; A61F 2240/001; A61F 2250/0098

(57) ABSTRACT

A stent may include a tubular body extending from a proximal end to a distal end and defining a lumen therethrough. The stent may comprise a scaffold structure formed from one or more filaments extending between the proximal and distal ends, and a polymeric coating disposed along at least a portion of the scaffold structure. A distal region of the tubular body includes a plurality of openings through the polymeric coating while maintaining the polymeric coating on the one or more filaments. The polymeric coating may comprise silicone and is configured to increase radial force of the tubular body.

### CROSS REFERENCE TO RELATED APPLICATIONS

The present application claims the benefit of U.S. Provisional Patent Application Ser. No. 63/753,010, filed Feb. 3, 2025, the disclosure of which is incorporated herein by reference.

### TECHNICAL FIELD

The present disclosure relates generally to methods and apparatuses for various ailments. More particularly, the disclosure relates to different configurations and methods of manufacture and use of a stent.

### BACKGROUND

Implantable stents are devices that are placed in a body structure, such as a blood vessel, esophagus, trachea, biliary tract, colon, intestine, stomach or body cavity, to provide support and to maintain patency of the structure or to maintain patency between two structures that have been combined via a stent for an alternate drainage path (e.g., transmural drainage). These devices are manufactured by any one of a variety of different manufacturing methods and may be used according to any one of a variety of methods for a variety of applications. Of the known medical stents, delivery systems, and methods, each has certain advantages and disadvantages. Thus, there is an ongoing need to provide alternative stent designs.

### SUMMARY

The disclosure is directed to several alternative designs, materials and methods of manufacturing medical device structures and assemblies, and the use thereof. An example medical device may include a stent.

In an example, a stent may include a tubular body extending from a proximal end to a distal end and defining a lumen therethrough, a scaffold structure formed from one or more filaments extending between the proximal end and the distal end, and a polymeric coating disposed along at least a portion of the scaffold structure, wherein a distal region of the tubular body may include a plurality of openings through the polymeric coating while maintaining the polymeric coating on the one or more filaments.

Alternatively or additionally to any of the examples above, in another example, the polymeric coating may be configured to increase radial force of the tubular body.

Alternatively or additionally to any of the examples above, in another example, the polymeric coating may comprise silicone.

Alternatively or additionally to any of the examples above, in another example, the distal region may extend along about 20% to 40% of a length of the tubular body.

Alternatively or additionally to any of the examples above, in another example, the openings of the plurality of openings may be spaced about a circumference of the tubular body.

Alternatively or additionally to any of the examples above, in another example, the plurality of openings may be formed through the polymeric coating after a coating process.

Alternatively or additionally to any of the examples above, in another example, the stent may further comprise at least one radiopaque marker positioned at a transition between the distal region and a fully coated region of the tubular body.

Alternatively or additionally to any of the examples above, in another example, the stent may further comprise one or more anti-migration elements projecting radially outwardly from the tubular body.

Alternatively or additionally to any of the examples above, in another example, the one or more anti-migration elements may include proximal anti-migration elements oriented towards the distal end of the stent and distal anti-migration elements oriented towards the proximal end of the stent.

Alternatively or additionally to any of the examples above, in another example, the stent may further comprise a reinforcing filament interwoven with the scaffold structure.

Alternatively or additionally to any of the examples above, in another example, the proximal end may include a proximal cage having an outer diameter greater than an outer diameter of the tubular body.

Alternatively or additionally to any of the examples above, in another example, the polymeric coating may increase frictional force between the tubular body and a body lumen.

In an example, a method of manufacturing a stent may include forming a tubular scaffold structure from one or more filaments, applying a polymeric coating to the tubular scaffold structure, and forming a plurality of openings through the polymeric coating in a distal region of the tubular scaffold structure while maintaining the polymeric coating on the one or more filaments.

Alternatively or additionally to any of the examples above, in another example, forming the plurality of openings may comprise laser ablation.

Alternatively or additionally to any of the examples above, in another example, the method may further comprise attaching at least one radiopaque marker at a transition between the distal region and a fully coated region.

In an example, a stent may include a tubular body extending from a proximal end to a distal end and defining a lumen therethrough, a scaffold structure formed from one or more filaments extending between the proximal end and the distal end, and a polymeric coating disposed along at least a portion of the scaffold structure, wherein a distal region of the tubular body may include a plurality of openings through the polymeric coating while maintaining the polymeric coating on the one or more filaments.

Alternatively or additionally to any of the examples above, in another example, the polymeric coating may be configured to increase radial force of the tubular body.

Alternatively or additionally to any of the examples above, in another example, the polymeric coating may comprise silicone.

Alternatively or additionally to any of the examples above, in another example, the distal region may extend along about 20% to 40% of a length of the tubular body.

Alternatively or additionally to any of the examples above, in another example, the openings of the plurality of openings may be spaced about a circumference of the tubular body.

Alternatively or additionally to any of the examples above, in another example, the plurality of openings may be formed through the polymeric coating after a coating process.

Alternatively or additionally to any of the examples above, in another example, the plurality of openings may be formed during the polymeric coating process.

Alternatively or additionally to any of the examples above, in another example, the stent may further comprise at least one radiopaque marker positioned at a transition between the distal region and a fully coated region of the tubular body.

Alternatively or additionally to any of the examples above, in another example, the stent may further comprise one or more anti-migration elements projecting radially outwardly from the tubular body.

Alternatively or additionally to any of the examples above, in another example, the one or more anti-migration elements may include proximal anti-migration elements oriented towards the distal end of the stent and distal anti-migration elements oriented towards the proximal end of the stent.

Alternatively or additionally to any of the examples above, in another example, the stent may further comprise a reinforcing filament interwoven with the scaffold structure.

Alternatively or additionally to any of the examples above, in another example, the proximal end may include a proximal cage having an outer diameter greater than an outer diameter of the tubular body.

In an example, a stent may include a tubular member formed from a single wire extending between a proximal end and a distal end, a plurality of circumferential segments formed by the single wire, a polymeric covering extending along at least a portion of the tubular member, and one or more anti-migration elements formed from the single wire, wherein a distal region of the tubular member may include a plurality of openings through the polymeric coating while maintaining the polymeric coating on the single wire.

Alternatively or additionally to any of the examples above, in another example, the one or more anti-migration elements may include proximal anti-migration elements oriented toward the second end and distal anti-migration elements oriented toward the proximal end.

Alternatively or additionally to any of the examples above, in another example, the stent may further comprise a reinforcing strip extending longitudinally along the tubular member.

Alternatively or additionally to any of the examples above, in another example, the polymeric covering may increase frictional force between the tubular member and a body lumen.

In an example, a method of manufacturing a stent may include forming a tubular scaffold structure from one or more filaments, applying a polymeric coating to the tubular scaffold structure, and forming a plurality of openings through the polymeric coating in a distal region of the tubular scaffold structure while maintaining the polymeric coating on the one or more filaments.

Alternatively or additionally to any of the examples above, in another example, forming the plurality of openings may comprise laser ablation.

Alternatively or additionally to any of the examples above, in another example, the method may further comprise attaching at least one radiopaque marker at a transition between the distal region and a fully coated region.

Alternatively or additionally to any of the examples above, in another example, applying the polymeric coating may comprise applying silicone.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1 is a partial cross-sectional view of an illustrative stent implanted in a body to create a pathway between an intrahepatic duct and the stomach.

FIG. 2 illustrates a side view of an illustrative implant in an expanded state.

FIG. 3A is a flat pattern view illustrating selected aspects of another illustrative implant.

FIG. 3B is a perspective view of the illustrative stent shown in FIG. 3A.

FIG. 4A illustrates a side view of another illustrative implant in an expanded state.

FIG. 4B is an enlarged view of a portion of the distal end region, taken at detail 4B of FIG. 4A.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings, in which like elements in different drawings are numbered in like fashion. The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

In some instances, it may be desirable to provide an endoluminal implant, or stent, that can deliver luminal patency within the pancreaticobiliary tree of a patient. The relatively narrow biliary tract ducts consist of a series of bifurcations linking the liver, gallbladder, and pancreas via the papilla to the duodenal space for the transportation of bile and related enzymic substances for many metabolic functions but most commonly the body's ability to digest and absorb fats and vitamins D and K. However, blockages may occur in the pancreaticobiliary tree due to tumor related duct narrowing, stricture formation, infections, or stone and sludge generation among other etiologies. Endoscopic retrograde cholangiopancreatography (ERCP) is used to diagnose and treat these duct narrowings, regardless of the malignant or benign nature of the disease. However, ERCP may not always be an option or may be unsuccessful in difficult anatomies or challenging disease states. Hepaticogastrostomy (HGS) may be used in cases where ERCP is not an option or fails. Other procedures, such as, but not limited to, choledochoduodenostomy (CDS) may be used to directly connect the common bile duct to the duodenum.

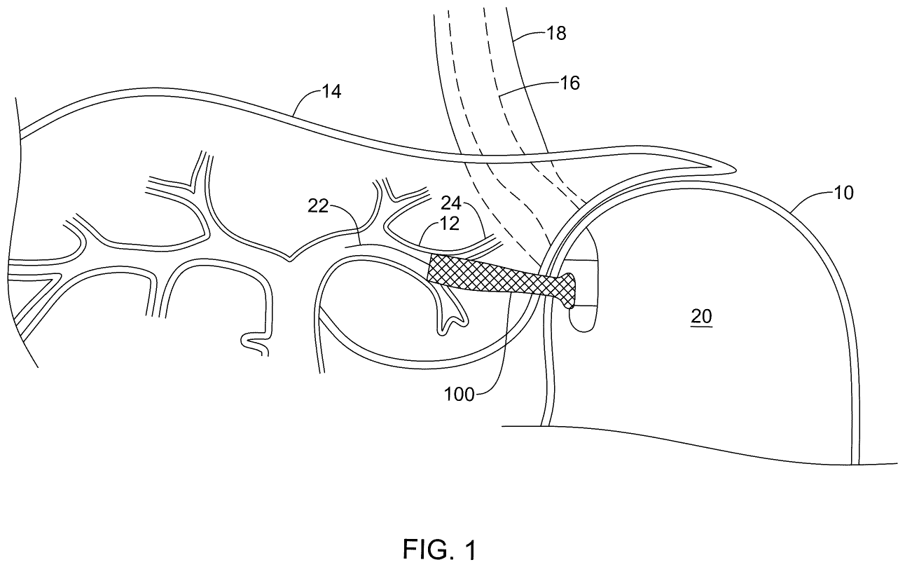

HGS is a procedure that targets an intrahepatic duct directly from the stomach and places a stent creating an artificial pathway (bridging the peritoneal cavity) to facilitate ongoing internal biliary drainage. FIG. 1 is a partial cross-sectional view of an illustrative stent 100 implanted in a body to create a pathway between an intrahepatic (bile) duct 12 and the stomach 10. The HGS procedure is completed using an endoscopic ultrasound scope or other endoscope 16 advanced through the esophagus 18 that allows the endoscopist to “see” through the wall of the stomach 10 and into the liver 14 prior to gaining access. The endoscopist may position the scope 16 with an appropriate clear trajectory to the intrahepatic duct 12. Guidewire access to the duct 12 is then made using a needle followed by enlargement of the tract using a balloon and/or cystotome. Alternatively, access is gained using a specialized all in one device. The guidewire 22 is advanced from the stomach 10 into the duct 12. Finally, a stent 100 is advanced and deployed to create and maintain the bridge between the duct 12 and an interior 20 of the stomach 10 thus facilitating ongoing internal biliary drainage. An additional factor endoscopists balance when positioning the endoscope 16 for this procedure is also the proximal positioning of the stent 100 within the stomach to minimize food impaction/migration into the stent lumen. Stent migration out of the stomach may be one of the highest severity risks with HGS stenting. Migration of the stent 100 from the stomach can result in leakage into the peritoneum causing biliary peritonitis. Additional concerns with traditional braided stents used in this indication is the stent foreshortening which may cause deployment accuracy issues. Further, it may be desirable for the stent 100 to prevent occlusion when placed across a side branch 24 of the biliary tree. The present disclosure is directed towards stents for use in HGS which inhibit stent migration, inhibit or limit stent foreshortening, and allow for drainage of side branches 24.

While the present disclosure is described with respect to an HGS procedure, the devices, systems, and/or methods described herein may be used in stents, endoluminal implants, or transluminal implants where the proximal end terminates in the upper GI tract. This may include devices placed during a CDS procedure or during a successful ERCP procedure, among others. Further, while the present disclosure is described with respect to the pancreaticobiliary ductal system, the devices, systems, and/or methods described herein may be used in stents, endoluminal implants, or transluminal implants positioned in other parts of the body, such as, but not limited to, bodily tissue, bodily organs, vascular lumens, non-vascular lumens and combinations thereof, such as, but not limited to, in the coronary or peripheral vasculature, trachea, bronchi, colon, small intestine, esophagus, biliary tract, urinary tract, prostate, brain, stomach, and the like.

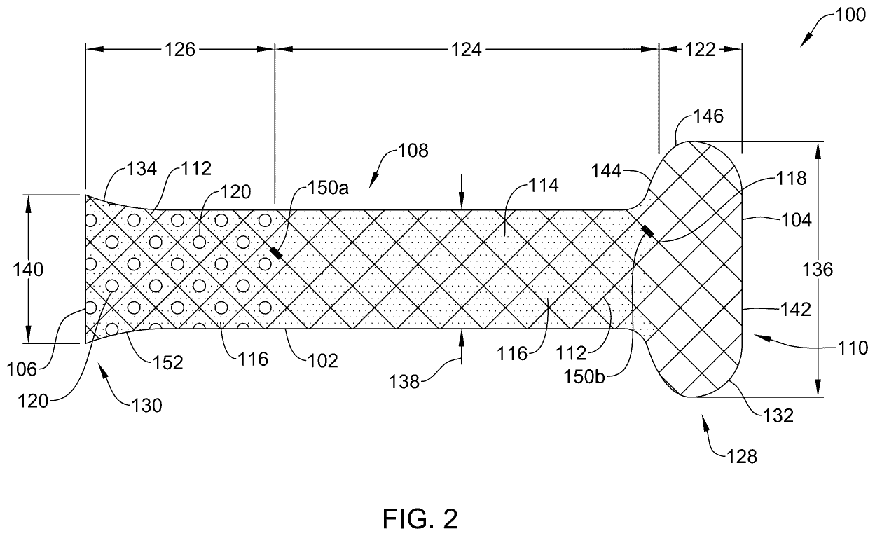

FIG. 2 illustrates a side view of an illustrative implant 100 that is adapted for use in fluidly coupling two different body lumens, such as, but not limited to, a stent, in an expanded state. In some instances, the stent 100 may be formed from an elongated tubular member 102. While the stent 100 is described as generally tubular, it is contemplated that the stent 100 may take any cross-sectional shape desired. The stent 100 may have a first, or proximal, end 104, a second, or distal, end 106, and an intermediate region 108 disposed between the first end 104 and the second end 106. The stent 100 may include a lumen 110 extending from a first opening adjacent the first end 104 to a second opening adjacent to the second end 106 to allow for the passage of bile, fluids, and the like therethrough.

The stent 100 may be radially expandable from a first radially collapsed configuration (not explicitly shown) to a second radially expanded configuration, as shown in FIG. 2. The stent 100 may be structured to extend across two non-adherent structures/tissues and to apply a radially outward pressure to create an opening or passage between the two non-adherent structures/tissues, thereby forming an anastomosis between the two separate anatomical structures.

The tubular member 102 of the stent 100 may have a scaffold structure, fabricated from one or more, or a plurality of interwoven filaments or struts 112. The scaffold structure may extend from the first end 104 to the second end 106 of the stent 100. For example, the scaffold structure, and thus the filament(s) thereof, may extend continuously from the first end 104 to the second end 106 of the stent 100. In some embodiments, the stent 100 may be formed with one filament interwoven with itself (e.g., knitted) to form the scaffold structure. In other embodiments, the stent 100 may be formed with several interwoven filaments (e.g., braided) to form the scaffold structure. Thus, in such instances one or more of the filament(s) forming the scaffold structure may extend continuously from the first end 104 to the second end 106 of the stent 100. In still another embodiment, the stent 100 may include a laser cut tubular member to form the scaffold structure. A laser cut tubular member may have an open and/or closed cell geometry including one or more interconnected struts formed as a monolithic structure from the tubular member. In such instances, the laser cut tubular member forming the scaffold structure may extend continuously from the first end 104 to the second end 106 of the stent 100.

In some instances, an inner and/or outer surface of the scaffold structure of the stent 100 may be entirely, substantially, or partially, covered with a polymeric covering or layer 114. For example, a covering or coating 114 may extend across the open cells of the scaffold structure to prevent tissue ingrowth into the lumen 110 of the stent 100. In some cases, the covering or coating 114 may prevent food or particulate ingress into the lumen 110 or fluid leakage from the lumen 110 along the coated regions. While not explicitly shown, the covering or coating 114 may include an outer layer disposed over an outer surface of the scaffold structure and/or an inner layer disposed over an inner (e.g., luminal) surface of the scaffold structure. In some embodiments, the stent 100 may include only an outer polymeric covering on an outer surface of the scaffold structure. In other embodiments, the stent 100 may include only an inner polymeric covering on an inner surface of the scaffold structure. In some instances, an inner layer and an outer layer may be formed as a single unitary structure to form the covering or coating 114. In other embodiments, an inner layer and an outer layer may be formed as separate layers to collectively form the covering or coating 114. The inner and outer layers may be formed from the same material or different materials, as desired. The covering or coating 114 may span or be disposed within openings or interstices 116 defined between adjacent stent filaments or struts 112 of the scaffold structure. It can be appreciated that as an inner layer and an outer layer extend outwardly and inwardly, respectively, they may touch and/or form an interface region within the spaces 116 (e.g., openings, cells, interstices) in the wall of the scaffold structure of the stent 100. For example, the inner and outer layers may extend into the openings 116 defined between adjacent stent struts 112 and form an interface region. Further, the inner and outer layers may additionally extend between adjacent filaments or struts 112, thereby filling any space between adjacent filaments or struts 112, and thus prevent tissue ingrowth into the lumen of the stent 100. The covering or coating 114 may extend along an entire length of the stent 100 from the first end 104 to the second end 106. However, this is not required. The covering or coating 114 may extend along less than an entire length of the stent 100.

In other embodiments, the covering or coating 114 may extend along only a portion of the stent 100 or the coating pattern may differ along a length of the stent 100. For example, the covering or coating 114 may extend from a first end 118 distal to the proximal end 104 of the stent 100 to the distal end 106 of the stent 100. One or more holes or apertures 120 may be formed through the covering or coating 114 along a distal end region 126 of the stent 100. The one or more apertures 120 may be formed through the covering or coating spanning the openings 116 between adjacent stent struts 112 while the struts 112 remain encapsulated or coated with the covering or coating 114. The stent 100 may include different coating patterns at different regions of the stent 100. The stent 100 may include a proximal end region 122 free from the covering or coating 114 (e.g., a first coating pattern), an intermediate fully coated region 124 (e.g., a second coating pattern), and a partially coated distal end region 126 (e.g., a third coating pattern). It is contemplated that the proximal end region 122 may remain uncoated to allow fluid or bile to pass through the lumen 110 of the stent 100 and exit the lumen 110 at the proximal end region 122 while restricting large particle ingress.

While the apertures 120 are illustrated as being generally circular, the apertures 120 may take any shape desired, such as, but not limited to, oblong, oval, square, rectangular, diamond, polygonal, eccentric, or the like. The apertures 120 may have an area that is less than an area of the openings 116 such that the covering or coating 114 is not removed from the struts 112. In some configurations, an opening or cell 116 may include more than one aperture 120. It is further contemplated that some of the cells or openings 116 within the distal end region 126 may be free from the apertures 120. The apertures 120 may be circumferentially and/or axially spaced in a uniform pattern or an eccentric arrangement, as desired.

The length of the partially coated distal end region 126 may be in the range of about 20% to 40% or about 30% of a nominal length of the stent 100. However, this is not required. The length of the partially coated distal end region 126 may be less than 20% of the nominal length of the stent 100 or greater than 40% of the nominal length of the stent 100, as desired. It is contemplated that the length of the uncoated proximal end region 122, the coated intermediate region 124, and/or the partially coated distal end region 126 may be determined based on the desired application, implant location, etc.

The apertures 120 may be formed after the stent 100 has been coated with the covering or coating 114. For example, the apertures 120 may be formed with laser ablation. It is contemplated that a laser may be computer controlled to allow for precise control over a size and/or position of apertures 120. In other embodiments, the apertures 120 may be formed with a punch or other tool. In yet other examples, the apertures 120 may be formed during the coating process using masking techniques or mandrels including raised regions having a height greater than the desired thickness of the covering or coating 114.

In some embodiments, the stent 100 may include one or more radiopaque markers 150a positioned at a proximal end of the partially coated distal end region 126. For example, the stent 100 may include one or more circumferentially spaced radiopaque markers 150a each positioned at a similar axial location to identify the start of the partially coated distal end region 126 or the transition between the fully coated intermediate region 124 and the partially coated distal end region 126. These radiopaque markers 150a may help the endoscopist position the stent 100 such that the partially coated distal end region 126 is spaced from the gap between the duct 12 and the stomach 10 to prevent leakage into the peritoneum. Said differently, the radiopaque markers 150a may help the endoscopist position the stent 100 such that a fully coated region 126 spans the gap between the duct 12 and the stomach 10. In some configurations, the stent 100 may include one or more radiopaque markers 150b positioned at a distal end of the uncoated proximal end region 122 or the transition between the uncoated proximal end region 122 and the fully coated intermediate region 124. For example, the stent 100 may include one or more circumferentially spaced radiopaque markers 150b each positioned at a similar axial location to identify the start of the fully coated intermediate region 124.

Generally, the tubular body 102 including the scaffold structure may be formed using a mandrel, laser cutting, or other technique generally known to one of skill in the art. The covering or coating 114 may be applied over the tubular body 102 using techniques such as, spray coating, dip methods, heat shrinking, or the like. If the stent 100 includes radiopaque marking elements 150a, 150b, the radiopaque marking elements 150a, 150b may be applied to the strut(s) 112 prior to applying the covering or coating 114. However, this is not required. If the stent 100 includes a reinforcing strip, the reinforcing strip may be interwoven with the scaffold structure prior to applying the covering or coating 114. After the covering or coating 114 is applied over the tubular body 102, the apertures 120 may be formed through the covering or coating 114 while maintaining the covering or coating 114 on the strut(s) 112. The apertures 120 may be formed using techniques such as laser ablation or mechanical cutting or punching. However, in some cases, the apertures 114 may be formed during the coating process.

The covering or coating 114 may be formed from silicone. However, other polymers may be used to form the covering or coating 114, as desired. Some illustrative polymers for use in the covering or coating 114 may include, but are not limited to, silicon carbide, poly(styrene-b-isobutylene-b-styrene) block copolymer (SIBS), poly(n-butyl methacrylate) (PBMA), poly(vinylidene fluoride)-hexafluoropropylene (PVDF-HFP), poly(lactide-co-glycolide) (PLGA), poly(ethylene-vinyl acetate) (PEVA), phosphorylcholine (PC), poly(lactic acid) (PLA), polyglycolic acid (PGA), poly(ethylene) (PE), polyurethanes (PUR), or the like.

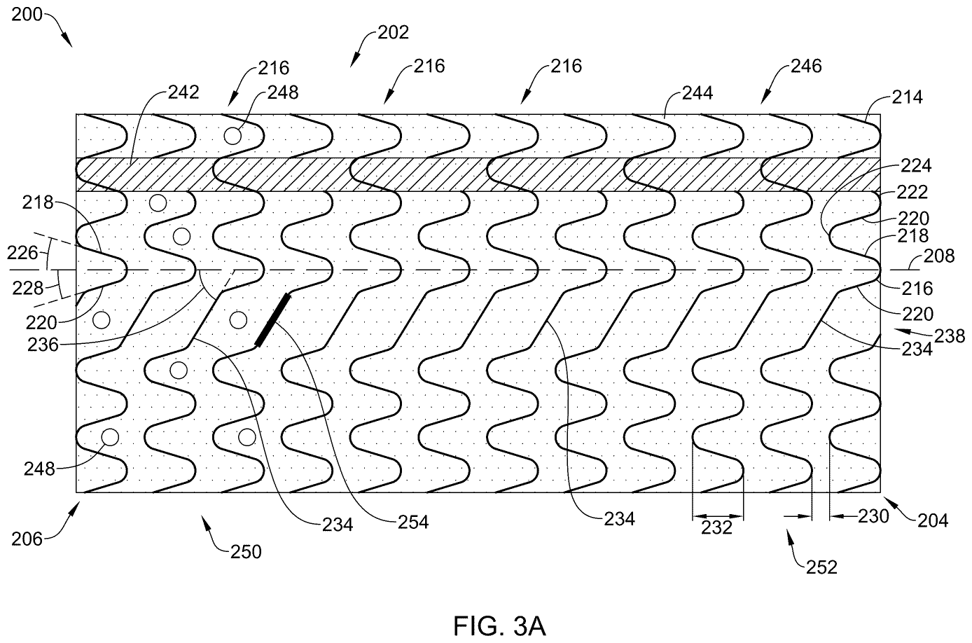

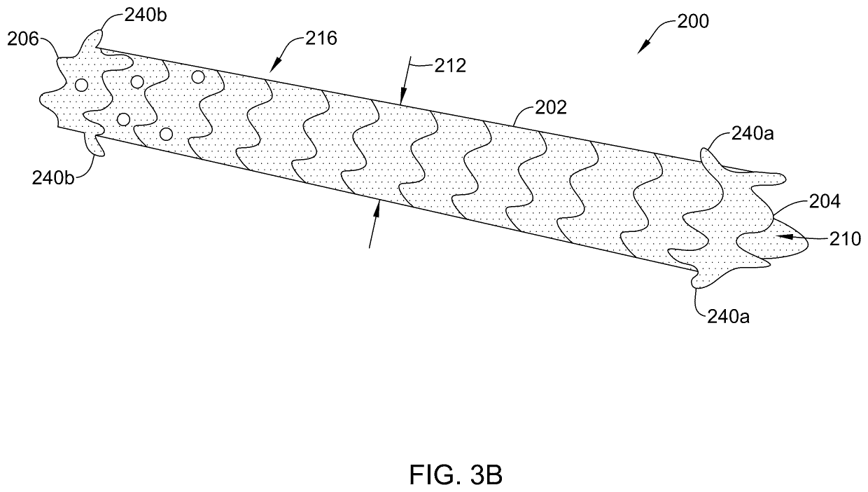

In addition to providing biliary drainage from the side ducts 24 through the apertures 120, the covering or coating 114 may reduce migration within the duct 12. It is contemplated that the covering or coating 114 on the stent 100 may increase the outward radial force of the stent 100 thereby increasing retention. For example, the covering or coating 114 may prevent or limit scissoring action in the struts 112 of the stent 100 at the cross-over points. Scissoring action may be the movement of the struts at the cross-over points which reduces an angle of the overlapping struts 112 at the cross-over point which in turn elongates the stent 100 and reduces an expanded diameter thereof. While not explicitly shown, the stent 100 may include a reinforcing filament similar in form and function to the reinforcing filament 242 described with respect to FIGS. 3A and 3B. Additionally, while not explicitly shown, the stent 100 may include one or more anti-migration elements similar in form and function to the anti-migration elements described with respect to FIGS. 3A and 3B.

It is contemplated that the scaffold structure, e.g., the filaments and/or struts, of the stent 100 can be made from a number of different materials such as, but not limited to, metals, metal alloys, shape memory alloys, and/or polymers, as desired, enabling the stent 100 to be expanded into shape when accurately positioned within the body. In some instances, the material may be selected to enable the stent 100 to be removed with relative ease as well. For example, the stent 100 can be formed from alloys such as, but not limited to, nitinol and Elgiloy®. Depending on the material selected for construction, the stent 100 may be self-expanding or require an external force to radially expand the stent 100. In some embodiments, filaments may be used to make the stent 100, which may be composite filaments, for example, having an outer shell made of nitinol and having a platinum core. It is further contemplated that the filaments of the stent 100 may be formed from polymers including, but not limited to, polyethylene terephthalate (PET).

In some instances, in the radially expanded configuration, the stent 100 may include a first end region 128 proximate to the first end 104 and a second end region 130 proximate to the second end 106. In some embodiments, the first end region 128 may include a proximal cage 132 positioned adjacent to the first end 104 and the second end region 130 may include a distal end region 134 positioned adjacent to the second end 106 of the stent 100. It is contemplated that the proximal cage 132 and/or the distal end region 134 may be formed at a same time as the elongated tubular member 102 of the stent 100. For example, the proximal cage 132 and/or the distal end region 134 may be formed from the one or more, or plurality of filaments or struts forming the scaffold structure that extend continuously from the first end 104 to the second end 106 of the stent 100 as the elongated tubular member 102 is woven, knit, laser cut, or the like. The proximal cage 132 may be configured to engage an interior portion of the wall of the body cavity or body lumen. For example, the proximal cage 132 may be positioned against an interior of a first body lumen (e.g., the wall of the stomach 10). The distal end region 134 may be configured to be positioned within another lumen separate (e.g., duct 12) from the first body lumen. The distal end region 134 may increase in diameter relative to the intermediate region 108 to exert a radially outward force on a surface of the lumen of the duct 12 to facilitate anchoring of the stent 100. Thus, the stent 100 may be positioned to traverse between two separate anatomical structures. For example, in FIG. 1 the stent 100 is positioned such that it extends between the stomach 10 and the intrahepatic duct 12. The proximal cage 132 may be positioned in the stomach 10 and the distal end region 134 may be positioned in the intrahepatic duct 12. The intermediate region 108 of the stent 100 extending between the proximal cage 132 and the distal end region 134 may extend through the wall of the stomach 10 and into the lumen of the intrahepatic duct 12, interconnecting the two anatomical structures. In some cases, at least a portion of the proximal cage 132 may contact an interior of the wall of the stomach 10 and/or the distal end region 134 may contact an inner surface of the intrahepatic duct 12. The proximal cage 132 and/or the distal end region 134 may anchor the stent 100 against the stomach wall and the intrahepatic duct 12 so that stent 100 is inhibited or prevented from migrating. However, this is not required.

The proximal cage 132 may have an outer diameter 136 that is greater than an outer diameter 138 of the intermediate region 108. The diameter 138 of the intermediate region 108 may be in the range of about 0.006 inches (0.152 millimeters) to about 0.010 inches (0.254 millimeters). In some examples, the proximal cage 132 may have a generally spheroid shape, such as, but not limited to, a generally oblate spheroid including a proximal end 142, a distal shoulder 144, and a curved side wall 146 extending between the proximal end 142 and the distal shoulder 144. It is contemplated that the proximal cage 132 may take other shapes, such as, but not limited to, spherical, hemi-spherical, rectangular prism, irregular, or the like. The distal shoulder 144 of the proximal cage 132 may extend radially from the intermediate region 108 of the elongated tubular member 102. It is contemplated that the transition from the cross-sectional area of the intermediate region 108 to the proximal cage 132 may be gradual, sloped, or occur in an abrupt step-wise manner, as desired. In some examples, the distal shoulder 144 may extend generally orthogonal to a longitudinal axis of the elongated tubular member 102. However, it is contemplated that the distal shoulder 144 may extend at non-orthogonal angles, as desired. The angle of the distal shoulder 144 may be selected based on the target anatomy. In some cases, the distal shoulder 144 may provide a mechanical stop between the proximal cage 132 and the target anatomy. The curved side wall 146 may have a curved semi-circular shape that gradually increases in cross-sectional dimension and then gradually decreases in cross-sectional dimension in a direction parallel to the longitudinal axis of the elongated tubular body 102. However, the curved side wall 146 may take other shapes including curved, linear, or irregular shapes, as desired.

The distal end region 134 may include a flare or may gradually increase in outer diameter from a proximal end 152 of the distal end region 134 having an outer diameter similar to or approximately equal to the outer diameter 138 of the intermediate region to a second outer diameter 140 adjacent to or at the distal end 106 of the stent 100. However, this is not required. In some examples, the distal end region 134 may have a same diameter as the diameter 138 of the intermediate region 108 along an entire length of the distal end region 134. In other examples, the distal end region 134 may include an abrupt or stair-step transition between a first outer diameter and the second outer diameter 140.

In some embodiments, the proximal cage 132 may have a first outer diameter 136 and the distal end region 134 may have a second outer diameter 140. The outer diameter 136 of the proximal cage 132 and/or the outer diameter 140 of the distal end region 134 may be greater than the outer diameter 138 of the intermediate region 108. The inner diameter of at least a portion of the proximal cage 132 and/or the distal end region 134 may be greater than the inner diameter of the intermediate region 108. In some instances, the first and second outer diameters 136, 140 may be approximately the same, while in other instances, the first and second outer diameters 136, 140 may be different. For example, in some cases, the outer diameter 136 of the proximal cage 132 may be greater than the outer diameter 140 of the distal end region 134. The stent 100 may take other shapes, as desired. In some embodiments, the distal end region 134 of the stent 100 may include a structure similar to the proximal cage 132. In other embodiments, the stent 100 may have a generally uniform outer diameter from the proximal end 104 to the distal end 106 thereof. It is contemplated that the outer diameter of the stent 100 may be varied to suit the desired application.

FIG. 3A is a flat pattern view illustrating selected aspects of another illustrative implant 200 that is adapted for use in fluidly coupling two different body lumens, such as, but not limited to, a stent. FIG. 3B is a perspective view of the illustrative stent 200. The stent 200 may comprise a body portion 202 extending axially from a first end 204 to a second end 206 along a central longitudinal axis 208. In some embodiments, the first end 204 and the second end 206 may define a length of the stent 200 and/or the body portion 202. In at least some embodiments, the body portion 202 may be an elongated tubular member (see, for example, FIG. 3B). The body portion 202 may define a lumen 210 extending therethrough from the first end 204 to the second end 206. The stent 200 may be radially expandable from a first radially collapsed configuration (not explicitly shown) to a second radially expanded configuration, as shown in FIG. 3B. The stent 200 may be structured to extend across two non-adherent structures/tissues and to apply a radially outward pressure to create an opening or passage between the two non-adherent structures/tissues, thereby forming an anastomosis between the two separate anatomical structures.

In some embodiments, the outer diameter 212 of the body portion 202 may be generally uniform and/or generally constant along the length of the stent 200 and/or the body portion 202. In some embodiments, the outer diameter 212 of the body portion 202 may be generally uniform and/or generally constant along the length of the body portion 202 except for a flared first end portion and/or a flared second end portion, where present. In some embodiments, the outer diameter 212 of the body portion 202 may be generally uniform and/or generally constant along the length of the body portion 202 from the flared first end portion to the flared second end portion, where present. In some embodiments, an outer diameter 212 of the body portion 202 can be in a range from about 4 to 40 millimeters (0.157 to 1.57 inches), among other possibilities. In some embodiments, the outer diameter 212 may be in a range from about 6 to 10 millimeters (0.236 to 0.394 inches). In some embodiments, the stent 200 may comprise a flared first end portion (not shown) and/or a flared second end portion (not shown), wherein an outer diameter of the flared first end portion is greater than an outer diameter 212 of the body portion 202 and/or an outer diameter of the flared second end portion is greater than the outer diameter 212 of the body portion 202. Other configurations are also contemplated.

In some embodiments, the stent 200 and/or the body portion 202 may be formed from a single wire 214 extending from the first end 204 to the second end 206. In some embodiments, the flared first end portion and/or the flared second end portion, where present, may be formed from the single wire 214. The single wire 214 may extend circumferentially around the central longitudinal axis 208 and/or the single wire 214 may encircle the central longitudinal axis 208. The single wire 214 is and/or consists of only one wire.

In some embodiments, the single wire 214 may form a plurality of circumferential segments 216 extending non-helically around the central longitudinal axis 208. In some embodiments, the plurality of circumferential segments 216 may be substantially parallel to each other. In some embodiments, each circumferential segment of the plurality of circumferential segments 216 may form a ring extending around the central longitudinal axis 208. Some suitable but non-limiting examples of materials for the stent 200 and/or the single wire 214, such as metallic materials, composite materials, shape memory materials, combinations thereof, etc., are discussed below. Illustrative arrangements of the single wire 214 are described in commonly assigned U.S. Patent Application Ser. No. 63/648,355, titled, ENDOPROSTHESIS WITH IMPROVED CHARACTERISTICS, the disclosure of which is hereby incorporated by reference.

In some embodiments, each circumferential segment of the plurality of circumferential segments 216 may comprise an undulating arrangement (e.g., a sinusoidal arrangement) of first struts 218 and second struts 220 defining peaks 222 and valleys 224. In at least some embodiments, within any particular circumferential segment of the plurality of circumferential segments 216, the first struts 218 have a first length defined by the peaks 222 and the valleys 224 and the second struts 220 have a second length defined by the peaks 222 and the valleys 224, wherein the first length is equal to the second length. In some embodiments, the first length and the second length may be uniform and/or constant among the plurality of circumferential segments 216. In some alternative embodiments, the first length and the second length may vary among the plurality of circumferential segments 216.

In some embodiments, the first struts 218 may be disposed at a first angle 226 relative to the central longitudinal axis 208 in a side view and/or a flat pattern view of the body portion 202 and/or the stent 200, and the second struts 220 may be disposed at a second angle 228 relative to the central longitudinal axis 208 in the side view and/or the flat pattern view of the body portion 202 and/or the stent 200. In at least some embodiments, the second angle may be different from the first angle. The first angle and the second angle can be in a range zero to 90 degrees, among other possibilities.

In some embodiments, each adjacent pair of circumferential segments of the plurality of circumferential segments 216 defines an axial spacing 230 therebetween. The axial spacing 230 may be measured axially and/or parallel to the central longitudinal axis 208. In some embodiments, the axial spacing 230 may be defined between the valleys 224 of one circumferential segment and the peaks 222 of an immediately adjacent circumferential segment. For the purpose of this disclosure, the axial spacing 230 shall be understood to not include any axial overlap with the first struts 218 and/or the second struts 220 of any given circumferential segment.

In some embodiments, the axial spacing 230 between adjacent pairs of circumferential segments of the plurality of circumferential segments 216 may be uniform along the length of the body portion 202 and/or the stent 200, as shown in FIG. 3A. In some embodiments, adjacent pairs of circumferential segments of the plurality of circumferential segments 216 are equally and/or uniformly spaced apart along the length of the body portion 202 and/or the stent 200. In some embodiments, the axial spacing 230 between adjacent pairs of circumferential segments of the plurality of circumferential segments 216 may vary along the length of the body portion 202 and/or the stent 200.

In some embodiments, the peaks 222 and the valleys 224 of each circumferential segment of the plurality of circumferential segments 216 may define a circumferential segment height 232 for that circumferential segment measured parallel to the central longitudinal axis 208. In some embodiments, the circumferential segment height 232 may be uniform along the length of the body portion 202 and/or the stent 200 and/or among the plurality of circumferential segments 216, as shown in FIG. 3A. The circumferential segment height 232 may vary along the length of the body portion 202 and/or the stent 200 and/or among the plurality of circumferential segments 216.

Each circumferential segment of the plurality of circumferential segments 216 may be connected to an axially adjacent circumferential segment of the plurality of circumferential segments 216 by a transition segment 234 of the single wire 214. In at least some embodiments, each circumferential segment of the plurality of circumferential segments 216 may be discontinuous and/or may extend around less than a full circumference of the stent 200 and/or the body portion 202. Instead, each circumferential segment of the plurality of circumferential segments 216 may have a first end and a second end, wherein the transition segment 234 extends between a first end of one circumferential segment of the plurality of circumferential segments 216 and a second end of an immediately adjacent circumferential segment of the plurality of circumferential segments 216 (or the transition segment 234 extends between a second end of one circumferential segment of the plurality of circumferential segments 216 and a first end of an immediately adjacent circumferential segment of the plurality of circumferential segments 216). In some embodiments, at least a portion of the first end circumferential segment 216 and/or the second end circumferential segment 216 may extend completely around the full circumference of the stent 200 and/or the body portion 202, and the at least one medial circumferential segment 216 may be discontinuous and/or may extend around less than the full circumference of the stent 200 and/or the body portion 202.

The transition segment 234 of the single wire 214 may be disposed at a third angle 236 relative to the central longitudinal axis 208 in the side view and/or the flat pattern view of the body portion 202 and/or the stent 200. In at least some embodiments, the third angle 236 may be different from the first angle 226 and the second angle 228. The lengths of the transition segment can be in a range from about 5 millimeters (0.197 inches) to about 20 millimeters (0.787 inches), in some instances. In some embodiments, each circumferential segment of the plurality of circumferential segments 216 may be connected to an axially adjacent circumferential segment of the plurality of circumferential segments 216 by a transition segment 234 of the single wire 214 disposed nonparallel to the central longitudinal axis 208, the first struts 218, and the second struts 220 in the side view and/or the flat pattern view of the body portion 202 and/or the stent 200. Other configurations are also contemplated.

In some embodiments, the transition segment 234 extending and/or disposed between axially adjacent circumferential segments of the plurality of circumferential segments 216 may extend between a valley 224 of one circumferential segment and a peak 222 of an immediately axially adjacent circumferential segment (or between a peak of one circumferential segment and a valley of an immediately axially adjacent circumferential segment). As illustrated, the transition segment(s) 234 extend from a peak 222 of a first circumferential segment disposed closer to the first end 204 to a valley 224 of a second circumferential segment immediately axially adjacent the first circumferential segment that is disposed closer to the second end 206 than the first circumferential segment. Other configurations are also contemplated. In some embodiments, the transition segment 234 may be disposed nonparallel with the first struts 218 and the second struts 220 of one circumferential segment (e.g., the first circumferential segment) and the first struts 218 and the second struts 220 of the immediately axially adjacent circumferential segment (e.g., the second circumferential segment).

In some embodiments, all transition segments (e.g., an array of transition segments consisting of each transition segment 234 connecting and/or extending between adjacent circumferential segments of the plurality of circumferential segments 216) of the single wire 214 may collectively define a transition zone 238. In some embodiments, the transition zone 238 may extend parallel to the central longitudinal axis 208, as shown in FIG. 3A. In some embodiments, the third angle 236 may be uniform along and/or within the transition zone 238. In some embodiments, each transition segment 234 of the single wire 214 may be aligned axially along the length of the body portion 202 and/or the stent 200. In some embodiments, the transition zone 238 may define a preferential bending plane for the stent 200 and/or the body portion 202 (e.g., the stent 200 and/or the body portion 202 may be configured to bend more easily along and/or within the transition zone 238 than in other directions). In some embodiments, the third angle 236 may vary along and/or within the transition zone 238. In some embodiments, the transition zone 238 may extend helically along and/or around the central longitudinal axis 208.

In some embodiments, the stent 200 and/or the body portion 202 may comprise one or more anti-migration elements 240a, 240b disposed along the length of the stent 200 and/or the body portion 202. In some embodiments, at least one circumferential segment of the plurality of circumferential segments 216 may comprise one or more anti-migration elements 240a, 240b projecting radially outwardly from the at least one circumferential segment and/or an outer surface of the stent 200 and/or the body portion 202 (see, for example, FIG. 3B). In some embodiments, at least one circumferential segment of the plurality of circumferential segments 216 may comprise a plurality of anti-migration elements 240a, 240b projecting radially outwardly from the at least one circumferential segment and/or the outer surface of the stent 200 and/or the body portion 202.

In some embodiments, anti-migration element(s) 240a positioned proximate the first end 204 may project radially outward (beyond the outer diameter 212) and toward the second end 206 (e.g., at a non-zero angle relative to the outer surface of the stent 200 and/or the body portion 202) and/or anti-migration element(s) 240b positioned proximate the second end 206 may project radially outward and toward the first end 204 (e.g., at a non-zero angle relative to the outer surface of the stent 200 and/or the body portion 202). In some embodiments, some anti-migration elements 240a positioned proximate the first end 204 may project toward the first end 204 and some may project the second end 206 (e.g., at a non-zero angle relative to the outer surface of the stent 200 and/or the body portion 202), and/or some anti-migration elements 240b positioned proximate the second end 206 may project toward the first end 204 and some may project the second end 206 (e.g., at a non-zero angle relative to the outer surface of the stent 200 and/or the body portion 202). Other configurations are also contemplated. The anti-migration elements 240a, 240b may be formed by bending or otherwise deforming the single wire 214. For example, one or more peaks 222 and/or valleys 224 may be bent radially outwards to form the anti-migration element(s).

The anti-migration element(s) 240a positioned proximate the first end 204 may be configured to engage a wall of stomach 10 when the stent 200 is deployed between the liver 14 and the stomach 10. The anti-migration element(s) 240a may be formed such that a valley 224 of a circumferential segment 216 is oriented or pointed towards the second end 206 of the stent 200. Thus, when deployed, distal movement of the stent 200 towards the liver 14 may cause the valley 224 of the anti-migration element(s) 240a positioned proximate the first end 204 to embed in, penetrate, or otherwise engage the wall of the stomach 10 to prevent or further limit distal movement of the stent 200.

The anti-migration element(s) 240b positioned proximate the second end 206 may be configured to engage the wall of the bile duct 12 when the stent 200 is deployed between the liver 14 and the stomach 10. The anti-migration element(s) 240b may be formed such that a peak 222 of a circumferential segment 216 is oriented or pointed towards the first end 204 of the stent 200. Thus, when deployed, proximal movement of the stent 200 towards the stomach 10 may cause the peak 222 of the anti-migration element(s) 240b positioned proximate the second end 206 to embed in, penetrate, or otherwise engage the wall of the bile duct 12 to prevent or further limit proximal movement of the stent 200.

The stent 200 may include any number of anti-migration element(s) 240a, 240b desired. For example, the stent 200 may include zero anti-migration element(s) 240a, 240b, one anti-migration element(s) 240a, 240b, or more than two anti-migration element(s) 240a, 240b, as desired. Further, the anti-migration element(s) 240a, 240b may be axially and/or circumferentially spaced in any configuration.

The stent 200 may comprise a reinforcing filament 242. In some embodiments, the reinforcing filament 242 may be formed of polyester, polytetrafluoroethylene (PTFE), or a combination thereof, among other suitable materials such as those detailed herein. In some embodiments, the reinforcing filament 242 may be formed of polyester. In some embodiments, the reinforcing filament 242 may be formed of PTFE.

The reinforcing filament 242 may have a uniform width taken along an entire length (e.g., extending longitudinally along the central longitudinal axis of the stent 200), as illustrated in FIG. 3A. However, in some embodiments, the reinforcing filament 242 can have a variable width (e.g., different widths taken at two or more positions along the length of the reinforcing filament 242).

The reinforcing filament 242 may have a uniform width and/or uniform thickness. For instance, the reinforcing filament 242 may have a uniform thickness (e.g., in the direction normal to the central longitudinal axis of the stent 200) along an entire length (e.g., in a direction along or coaxial with the central longitudinal axis of the stent 200) of the reinforcing filament. However, in some embodiments, the reinforcing filament 242 can have a variable thickness (e.g., different thicknesses at two or more positions along the length of the reinforcing filament 242).

The reinforcing filament 242 may be formed of a different material than a material of another portion of the stent 200 (e.g., a different than a material of the body portion 202 and/or than a material of the polymeric covering 244). For instance, the reinforcing filament 242 may be formed of a different material than at least the body portion 202 of the stent 200. Thus, the reinforcing filament 242 may have a different material property (e.g., a higher tensile strength) than a corresponding material property of the body portion 202 of the stent 200. For example, the reinforcing filament may be formed of a different material (e.g., a material having a higher tensile strength) than a material of the single wire 214 forming the body portion 202 of the stent 200. Thus, the presence of the reinforcing filament 242 may alter the overall properties of the stent 200, for example, increasing an overall tensile strength of the stent 200, thereby improving the characteristics of the stent 200 (e.g., mitigating any foreshortening of the stent 200). For example, the presence of the reinforcing filament may prevent any elongation when the stent 200 is constrained for delivery, and thereby prevent any subsequent foreshortening of the stent 200 when the stent 200 is radially expanded at a delivery location. In some embodiments, the reinforcing filament 242 may have a tensile strength of about 500 pounds per square inch or more, about 1,000 pounds per square inch or more, about 1,500 pounds per square inch or more, or about 2,000 pounds per square inch or more, for example. In certain embodiments, the reinforcing filament 242 may possess a tensile strength several orders of magnitude greater than that of the polymeric covering 244. For instance, the reinforcing filament 242 could exhibit a tensile strength at least two, three, four, five, six, seven, eight, nine, or ten times greater than that of the polymeric covering 244. For example, in some embodiments, the tensile strength of the reinforcing filament 242 may be in a range from about 2,000 to about 3,000 pounds per square inch, while that of the polymeric covering 244 may be in a range from about 200 to about 300 pounds per square inch, among other possibilities.

In some embodiments, the reinforcing filament 242 may be manifested as an elongated planar strip, as illustrated in FIG. 3A. Having the reinforcing filament 242 be manifested as an elongate planar strip can promote aspects herein (e.g., mitigation of any foreshortening experienced by the stent 200), and yet can maintain a relatively small or thin profile of the stent 200. However, other configurations of the reinforcing filament 242 are possible. For instance, in some embodiments, the reinforcing filament 242 can be manifested as an elongate rod or other structure that can be interwoven with plurality of circumferential segments 216 of the single wire 214. While FIG. 3A illustrates the presence of an individual reinforcing filament 242, in some embodiments, the stent 200 can include a plurality of reinforcing filaments 242.

The presence of the reinforcing filament 242 can promote aspects herein such as promoting mitigation of any foreshortening experienced by the stent 200, promoting conformability (e.g., to body lumen) of the stent 200, and/or mitigating any forces (e.g., axial and/or radial forces) imparted on the stent 200. As illustrated in FIG. 3A, the reinforcing filament 242 can extend substantially longitudinally along the body portion 202. For instance, the reinforcing filament 242 may extend continuously and substantially longitudinally along some or all of a length (in the same direction as the central longitudinal axis 208) of the body portion 202. For example, in some embodiments the reinforcing filament 242 may have a length that is substantially equal to at least half a length of the body portion 202. However, in some embodiments the reinforcing filament 242 may have a length that is substantially equal to a length of the body portion 202, as illustrated in FIG. 3A. Having a length of the reinforcing filament 242 be substantially equal to a length of the body portion 202 can promote aspects herein such as promoting mitigation of any foreshortening experienced by the stent 200, promoting conformability of the stent 200, and/or mitigating any forces (e.g., axial and/or radial forces) imparted on the stent 200 along an entire length of the stent 200.

In some embodiments, the reinforcing filament 242 may be interwoven with the single wire 214. For example, the reinforcing filament 242 may be interwoven with two or more of the plurality of circumferential segments 216 of the single wire 214. For instance, as illustrated in FIG. 3A, the reinforcing filament 242 may be interwoven in an alternating over and under fashion with the plurality of circumferential segments 216 of the single wire 214.

In some instances, an inner and/or outer surface of the scaffold structure of the stent 200 may be entirely, substantially or partially, covered with a polymeric covering or layer 244. For example, a covering or coating 244 may extend across the open cells (e.g., between adjacent circumferential segments 216) of the scaffold structure to prevent tissue ingrowth into the lumen 210 of the stent 200. In some cases, the covering or coating 244 may prevent food or particulate ingress into the lumen 210 or fluid leakage from the lumen 210 along the coated regions. While not explicitly shown, the covering or coating 244 may include an outer layer disposed over an outer surface of the scaffold structure and/or an inner layer disposed over an inner (e.g., luminal) surface of the scaffold structure. In some embodiments, the stent 200 may include only an outer polymeric covering on an outer surface of the scaffold structure. In other embodiments, the stent 200 may include only an inner polymeric covering on an inner surface of the scaffold structure. In some instances, an inner layer and an outer layer may be formed as a single unitary structure to form the covering or coating 244. In other embodiments, an inner layer and an outer layer may be formed as separate layers to collectively form the covering or coating 244. The inner and outer layers may be formed from the same material or different materials, as desired. The covering or coating 244 may span or be disposed within openings or interstices 246 defined between adjacent circumferential segments 216 of the scaffold structure. It can be appreciated that as an inner layer and an outer layer extend outwardly and inwardly, respectively, they may touch and/or form an interface region within the spaces (e.g., openings, cells, interstices) in the wall of the scaffold structure of the stent 200. For example, the inner and outer layers may extend into the openings 246 defined between adjacent circumferential segments 216 and form an interface region. Further, the inner and outer layers may additionally extend between adjacent circumferential segments 216, thereby filling any space between adjacent circumferential segments 216 and any space between first and second struts 218, 220, and thus prevent tissue ingrowth into the lumen of the stent 200. The covering or coating 244 may extend along an entire length of the stent 200 from the first end 204 to the second end 206. In other embodiments, the covering or coating 244 may extend along only a portion of the stent 200 or the coating pattern may differ along a length of the stent 200.

The stent 200 may include different coating patterns at different regions of the stent 200. For example, one or more holes or apertures 248 may be formed through the covering or coating 244 along a distal end region 250 (e.g., adjacent the second end 206) of the stent 200. The one or more apertures 248 may be formed through the covering or coating spanning the openings 246 between adjacent circumferential segments 216 while the circumferential segments 216 and the single wire 214 remain encapsulated or coated with the covering or coating 244. The stent 200 may include a fully coated region (e.g., a first coating pattern) and a partially coated distal end region 250 (e.g., a second coating pattern). It is contemplated that the proximal end region 252 may remain uncoated (not explicitly shown) to allow fluid or bile to pass through the lumen 210 of the stent 200 and exit the lumen 210 at the proximal end region 252 while restricting large particle ingress. However, this is not required. In some cases, the covering or coating 244 may extend from the first end 204 to a proximal end of the distal end region 250.

While the apertures 248 are illustrated as being generally circular, the apertures 248 may take any shape desired, such as, but not limited to, oblong, oval, square, rectangular, diamond, polygonal, eccentric, or the like. The apertures 248 may have an area that is less than an area of the openings 246 such that the covering or coating 244 is not removed from the single wire 214. In some configurations, an opening or cell 246 may include more than one aperture 248. It is further contemplated that some of the cells or openings 246 within the distal end region 250 may be free from the apertures 248. The apertures 248 may be circumferentially and/or axially spaced in a uniform pattern or an eccentric arrangement, as desired.

The length of the partially coated distal end region 250 may be in the range of about 20% to 40% or about 30% of a nominal length of the stent 200. However, this is not required. The length of the partially coated distal end region 250 may be less than 20% of the nominal length of the stent 200 or greater than 40% of the nominal length of the stent 200, as desired. It is contemplated that the length of an uncoated region (if so provided), the coated proximal end region 252 and/or the partially coated distal end region 250 may be determined based on the desired application, implant location, etc.

The apertures 248 may be formed after the stent 200 has been coated with the covering or coating 244. For example, the apertures 248 may be formed with laser ablation. It is contemplated that a laser may be computer controlled to allow for precise control over a size and/or position of apertures 248. In other embodiments, the apertures 248 may be formed with a punch or other tool. In yet other examples, the apertures 248 may be formed during the coating process using masking techniques or mandrels including raised regions having a height greater than the desired thickness of the covering or coating 244.

In some embodiments, the stent 200 may include one or more radiopaque markers 254 positioned at a proximal end of the partially coated distal end region 250. For example, the stent 200 may include one or more circumferentially spaced radiopaque markers 254 each positioned at a similar axial location to identify the start of the partially coated distal end region 250. These radiopaque markers 254 may help the endoscopist position the stent 200 such that the partially coated distal end region 250 is spaced from the gap between the duct 12 and the stomach 10 to prevent leakage into the peritoneum.

Generally, the tubular body 202 including the scaffold structure may be formed using a mandrel, laser cutting, or other technique generally known to one of skill in the art. The covering or coating 244 may be applied over the tubular body 200 using techniques such as, spray coating, dip methods, heat shrinking, or the like. If the stent 200 includes radiopaque marking elements 254, the radiopaque marking elements 254 may be applied to the single wire 214 prior to applying the covering or coating 244. However, this is not required. If the stent 200 includes a reinforcing strip 242, the reinforcing strip 242 may be interwoven with the scaffold structure prior to applying the covering or coating 244. After the covering or coating 244 is applied over the tubular body 202, the apertures 248 may be formed through the covering or coating 244 while maintaining the covering or coating 244 on the single wire 214. The apertures 244 may be formed using techniques such as laser ablation, mechanical cutting, or punching. However, in some cases, the apertures 244 may be formed during the coating process.

The covering or coating 244 may be formed from silicone. However, other polymers may be used to form the covering or coating 244, as desired. Some illustrative polymers for use in the covering or coating 244 may include, but are not limited to, silicon carbide, poly(styrene-b-isobutylene-b-styrene) block copolymer (SIBS), poly(n-butyl methacrylate) (PBMA), poly(vinylidene fluoride)-hexafluoropropylene (PVDF-HFP), poly(lactide-co-glycolide) (PLGA), poly(ethylene-vinyl acetate) (PEVA), phosphorylcholine (PC), poly(lactic acid) (PLA), polyglycolic acid (PGA), poly(ethylene) (PE), polyurethanes (PUR), or the like.

In addition to providing biliary drainage from the side ducts 24 through the apertures 248, the covering or coating 244 may reduce migration within the duct 12. It is contemplated that the covering or coating 244 on the stent 200 may increase the outward radial force of the stent 200 thereby increasing retention.

It is contemplated that the scaffold structure, e.g., the filaments and/or struts, of the stent 200 can be made from a number of different materials such as, but not limited to, metals, metal alloys, shape memory alloys, and/or polymers, as desired, enabling the stent 200 to be expanded into shape when accurately positioned within the body. In some instances, the material may be selected to enable the stent 200 to be removed with relative ease as well. For example, the stent 200 can be formed from alloys such as, but not limited to, nitinol and Elgiloy®. Depending on the material selected for construction, the stent 200 may be self-expanding or require an external force to radially expand the stent 200. In some embodiments, filaments may be used to make the stent 200, which may be composite filaments, for example, having an outer shell made of nitinol and having a platinum core. It is further contemplated that the filaments of the stent 200 may be formed from polymers including, but not limited to, polyethylene terephthalate (PET).

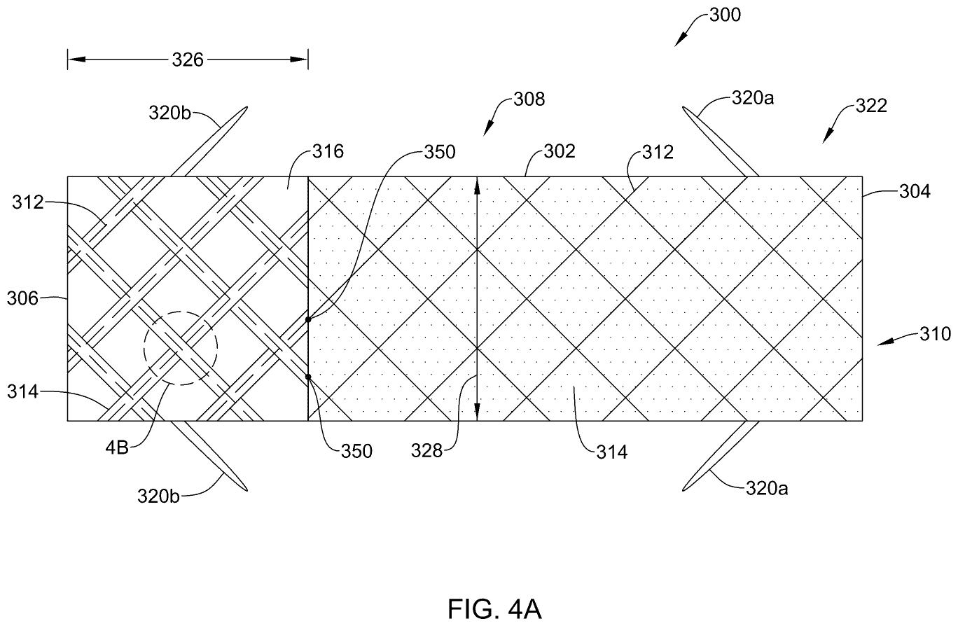

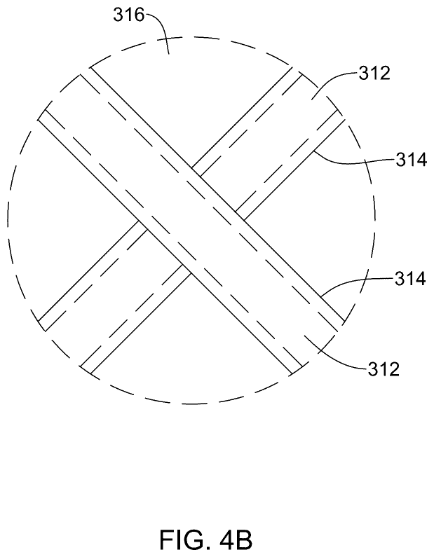

FIG. 4A illustrates a side view of an illustrative implant 300 that is adapted for use in fluidly coupling two different body lumens, such as, but not limited to, a stent, in an expanded state. In some instances, the stent 300 may be formed from an elongated tubular member 302. While the stent 300 is described as generally tubular, it is contemplated that the stent 300 may take any cross-sectional shape desired. The stent 300 may have a first, or proximal, end 304, a second, or distal, end 306, and an intermediate region 308 disposed between the first end 304 and the second end 306. The stent 300 may include a lumen 310 extending from a first opening adjacent the first end 304 to a second opening adjacent to the second end 306 to allow for the passage of bile, fluids, and the like therethrough.

The stent 300 may be radially expandable from a first radially collapsed configuration (not explicitly shown) to a second radially expanded configuration, as shown in FIG. 4A. The stent 300 may be structured to extend across two non-adherent structures/tissues and to apply a radially outward pressure to create an opening or passage between the two non-adherent structures/tissues, thereby forming an anastomosis between the two separate anatomical structures.

The tubular member 302 of the stent 300 may have a scaffold structure, fabricated from one or more, or a plurality of interwoven filaments or struts 312. The scaffold structure may extend from the first end 304 to the second end 306 of the stent 300. For example, the scaffold structure, and thus the filament(s) thereof, may extend continuously from the first end 304 to the second end 306 of the stent 300. In some embodiments, the stent 300 may be formed with one filament interwoven with itself (e.g., knitted) to form the scaffold structure. In other embodiments, the stent 300 may be formed with several interwoven filaments (e.g., braided) to form the scaffold structure. Thus, in such instances one or more of the filament(s) forming the scaffold structure may extend continuously from the first end 304 to the second end 306 of the stent 300. In still another embodiment, the stent 300 may include a laser cut tubular member to form the scaffold structure. A laser cut tubular member may have an open and/or closed cell geometry including one or more interconnected struts formed as a monolithic structure from the tubular member. In such instances, the laser cut tubular member forming the scaffold structure may extend continuously from the first end 304 to the second end 306 of the stent 300.

In some instances, an inner and/or outer surface of the scaffold structure of the stent 300 may be entirely, substantially, or partially, covered with a polymeric covering or layer 314. For example, a covering or coating 314 may extend across the open cells of the scaffold structure to prevent tissue ingrowth into the lumen 310 of the stent 300. In some cases, the covering or coating 314 may prevent food or particulate ingress into the lumen 310 or fluid leakage from the lumen 310 along the coated regions. While not explicitly shown, the covering or coating 314 may include an outer layer disposed over an outer surface of the scaffold structure and/or an inner layer disposed over an inner (e.g., luminal) surface of the scaffold structure. In some embodiments, the stent 300 may include only an outer polymeric covering on an outer surface of the scaffold structure. In other embodiments, the stent 300 may include only an inner polymeric covering on an inner surface of the scaffold structure. In some instances, an inner layer and an outer layer may be formed as a single unitary structure to form the covering or coating 314. In other embodiments, an inner layer and an outer layer may be formed as separate layers to collectively form the covering or coating 314. The inner and outer layers may be formed from the same material or different materials, as desired. The covering or coating 314 may span or be disposed within at least some of the openings or interstices 316 defined between adjacent stent filaments or struts 312 of the scaffold structure. It can be appreciated that as an inner layer and an outer layer extend outwardly and inwardly, respectively, they may touch and/or form an interface region within the spaces (e.g., openings, cells, interstices) in the wall of the scaffold structure of the stent 300. For example, the inner and outer layers may extend into the openings 316 defined between adjacent stent struts 312 and form an interface region. Further, the inner and outer layers may additionally extend between adjacent filaments or struts 312, thereby filling any space between at least some of the adjacent filaments or struts 312, and thus prevent tissue ingrowth into the lumen of the stent 300. The covering or coating 314 may extend along an entire length of the stent 300 from the first end 304 to the second end 306. However, this is not required. The covering or coating 314 may extend along less than an entire length of the stent 300. In other embodiments, the covering or coating 314 may extend along only a portion of the stent 300 or the coating pattern may differ along a length of the stent 300. For example, while not explicitly shown, a proximal end region 322 of the stent 300 may be free from the covering or coating 314 in a similar manner to the stent 100 described herein. It is contemplated that the proximal end region 322 may remain uncoated to allow fluid or bile to pass through the lumen 310 of the stent 300 and exit the lumen 310 at the proximal end region 322 while restricting large particle ingress.

The stent 300 may include different coating patterns at different regions of the stent 300. Along a proximal end region 322 of the stent 300, the covering or coating 314 may span across the open cells 316 to fully coat the tubular body 302 (e.g., a first coating pattern which fully encloses the lumen 310 along the circumferential surface). Along a distal end region 326 of the stent 300, the openings 316 may be substantially free from the covering or coating 314 while the strut(s) 312 are coated (e.g., a second coating pattern). FIG. 4B is an enlarged view of a portion of the distal end region 326, taken at detail 4B of FIG. 4A. The struts 312 of the stent 300 are coated with the covering or coating 314 while leaving the cells 316 of the stent 300 open. The covering or coating 314 around the struts 312 may act to elastically to resist deformation of the struts 312 thereby increasing the radial forces that press outward on the lumen of the duct 12. Additionally, the covering or coating 314 may increase the frictional force between the wall of the duct 12 and the stent 300. Increasing both the outward force acting on the wall of the duct 12 and the frictional force of the stent 300 may result in an increase in its retention within the duct 12. The covering or coating 314 may prevent or limit scissoring action in the struts 312 of the stent 300 at the cross-over points. Scissoring action may be the movement of the struts at the cross-over points which reduces an angle of the overlapping struts 312 at the cross-over point which in turn elongates the stent 300 and reduces an expanded diameter thereof. While not explicitly shown, the stent 300 may include a reinforcing filament similar in form and function to the reinforcing filament 242 described with respect to FIGS. 3A and 3B. Further, the uncoated cells 316 may allow fluid from the side branches 24 to drain into the lumen 310 of the stent 300.

Returning to FIG. 4A, in some configurations, the covering or coating 314 may be applied to the distal end region 326 of the stent 300 after the tubular body 302 has been formed. For example, a mandrel used to form the tubular body 302 may be removed from the distal end region 326 of the stent 300 during the coating process. This may allow the covering or coating 314 to drain from the openings 316 while allowing the covering or coating 314 to be applied to the struts 312. The struts 312 may be encapsulated or coated with the covering or coating 314. The covering or coating 314 may be applied over the tubular body 302 using techniques such as, spray coating, dip methods, heat shrinking, or the like. If the stent 300 includes radiopaque marking elements 350, the radiopaque marking elements 350 may be applied to the strut(s) 312 prior to applying the covering or coating 314. However, this is not required. If the stent 300 includes a reinforcing strip, the reinforcing strip may be interwoven with the scaffold structure prior to applying the covering or coating 314.

The length of the partially coated distal end region 326 may be in the range of about 20% to 40% or about 30% of a nominal length of the stent 300. However, this is not required. The length of the partially coated distal end region 326 may be less than 20% of the nominal length of the stent 300 or greater than 40% of the nominal length of the stent 300, as desired. It is contemplated that the length of fully coated portion of the stent 300 may be determined based on the length of the partially coated distal end region 326 and/or a length of an uncoated proximal end region 322 (if so provided).

In some embodiments, the stent 300 may include one or more radiopaque markers 350 positioned at a proximal end of the partially coated distal end region 326. For example, the stent 300 may include one or more circumferentially spaced radiopaque markers 350 each positioned at a similar axial location to identify the start of the partially coated distal end region 326. These radiopaque markers 350 may help the endoscopist position the stent 300 such that the partially coated distal end region 326 is distally spaced from the gap between the duct 12 and the stomach 10 to prevent leakage into the peritoneum. Said differently, the radiopaque markers 350 may help the endoscopist position the stent 300 such that a fully coated region spans the gap between the duct 12 and the stomach 10. In some configurations, the stent 300 may include one or more radiopaque markers (not explicitly shown) positioned at a distal end of the uncoated proximal end region 322. For example, the stent 300 may include one or more circumferentially spaced radiopaque markers each positioned at a similar axial location to identify the start of the fully coated region when a proximal uncoated region is provided.

The covering or coating 314 may be formed from silicone. However, other polymers may be used to form the covering or coating 314, as desired. Some illustrative polymers for use in the covering or coating 314 may include, but are not limited to, silicon carbide, poly(styrene-b-isobutylene-b-styrene) block copolymer (SIBS), poly(n-butyl methacrylate) (PBMA), poly(vinylidene fluoride)-hexafluoropropylene (PVDF-HFP), poly(lactide-co-glycolide) (PLGA), poly(ethylene-vinyl acetate) (PEVA), phosphorylcholine (PC), poly(lactic acid) (PLA), polyglycolic acid (PGA), poly(ethylene) (PE), polyurethanes (PUR), or the like.

It is contemplated that the scaffold structure, e.g., the filaments and/or struts, of the stent 300 can be made from a number of different materials such as, but not limited to, metals, metal alloys, shape memory alloys, and/or polymers, as desired, enabling the stent 300 to be expanded into shape when accurately positioned within the body. In some instances, the material may be selected to enable the stent 300 to be removed with relative ease as well. For example, the stent 300 can be formed from alloys such as, but not limited to, nitinol and Elgiloy®. Depending on the material selected for construction, the stent 300 may be self-expanding or require an external force to radially expand the stent 300. In some embodiments, filaments may be used to make the stent 300, which may be composite filaments, for example, having an outer shell made of nitinol and having a platinum core. It is further contemplated that the filaments of the stent 300 may be formed from polymers including, but not limited to, polyethylene terephthalate (PET).

In some embodiments, the stent 300 and/or the body portion 302 may comprise one or more anti-migration elements 320a, 320b disposed along the length of the stent 300 and/or the body portion 302. The one or more anti-migration elements 320a, 320b may project radially outwardly from an outer surface of the stent 300 and/or the body portion 302 (see, for example, FIG. 4A). In some embodiments, anti-migration element(s) 320a positioned proximate the first end 304 may project radially outward (beyond the outer diameter 328) and toward the second end 306 (e.g., at a non-zero angle relative to the outer surface of the stent 300 and/or the body portion 302) and/or anti-migration element(s) 320b positioned proximate the second end 306 may project radially outward and toward the first end 304 (e.g., at a non-zero angle relative to the outer surface of the stent 300 and/or the body portion 302). The anti-migration elements 320a, 320b may be formed by bending or otherwise deforming the strut(s) 312. Alternatively, or additionally, the anti-migration elements 320a, 320b may be separate members fixedly coupled to the strut(s) 312 through techniques such as, but not limited to, welding, soldering, adhesives, or the like.

The anti-migration element(s) 320a positioned proximate the first end 304 may be configured to engage a wall of stomach 10 when the stent 300 is deployed between the liver 14 and the stomach 10. The anti-migration element(s) 320a may be formed such that anti-migration element(s) 320a is oriented or pointed towards the second end 306 of the stent 300. Thus, when deployed, distal movement of the stent 300 towards the liver 14 may cause the anti-migration element(s) 320a positioned proximate the first end 304 to embed in, penetrate, or otherwise engage the wall of the stomach 10 to prevent or further limit distal movement of the stent 300.

The anti-migration element(s) 320b positioned proximate the second end 306 may be configured to engage the wall of the bile duct 12 when the stent 300 is deployed between the liver 14 and the stomach 10. The anti-migration element(s) 320b may be formed such that the anti-migration element(s) 320b is oriented or pointed towards the first end 304 of the stent 300. Thus, when deployed, proximal movement of the stent 300 towards the stomach 10 may cause anti-migration element(s) 320b positioned proximate the second end 306 to embed in, penetrate, or otherwise engage the wall of the bile duct 12 to prevent or further limit proximal movement of the stent 300.

The stent 300 may include any number of anti-migration element(s) 320a, 320b desired. For example, the stent 300 may include zero anti-migration element(s) 320a, 320b, one anti-migration element(s) 320a, 320b, or more than two anti-migration element(s) 320a, 320b, as desired. Further, the anti-migration element(s) 320a, 320b may be axially and/or circumferentially spaced in any configuration.

In some embodiments, anti-migration element(s) 320a positioned proximate the first end 304 may project radially outward and toward the first end 304 (e.g., at a non-zero angle relative to the outer surface of the stent 300 and/or the body portion 302) and/or anti-migration element(s) 320b positioned proximate the second end 306 may project radially outward and toward the second end 306 (e.g., at a non-zero angle relative to the outer surface of the stent 300 and/or the body portion 302). In some embodiments, some anti-migration elements 320a positioned proximate the first end 304 may project toward the first end 304 and some may project the second end 306 (e.g., at a non-zero angle relative to the outer surface of the stent 300 and/or the body portion 302), and/or some anti-migration elements 320b positioned proximate the second end 306 may project toward the first end 304 and some may project the second end 306 (e.g., at a non-zero angle relative to the outer surface of the stent 300 and/or the body portion 302). Other configurations are also contemplated.

In some configurations the tubular body 302 of the stent 300 may have a generally constant outer diameter 328 (excluding the anti-migration elements 320a, 320b) extending from the first end 304 to the second end 306. However, this is not required. In some cases, the outer diameter of the stent 300 may include a flare or may gradually increase in outer diameter from a point proximal to the second end 306 to the second end 306. Additionally, or alternatively, the outer diameter of the stent 300 may include a flare or may gradually increase in outer diameter from a point distal to the first end 304 to the first end 304. In other examples, the distal end region 326 and/or the proximal end region 322 may include an abrupt or step-wise transition between a first outer diameter and a second outer diameter. In some configurations, the proximal end region 322 may include a proximal cage similar in form and function to the proximal cage 132 described herein.

The materials that can be used for the various components of the medical stent(s) and the various elements thereof disclosed herein may include those commonly associated with medical devices. For simplicity purposes, the following discussion refers to the apparatus. However, this is not intended to limit the devices and methods described herein, as the discussion may be applied to other elements, members, components, or devices disclosed herein, such as, but not limited to, the medical stent, the filaments, the covering, flexible sleeve, and/or elements or components thereof.

In some instances, the apparatus, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS:
N10276 such as HASTELLOY® C276®, other HASTELLOY® alloys, and the like), nickel-copper alloys (e.g., UNS: N04400 such as MONEL® 400, NICKELVAC® 400, NICORROS®400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some instances, portions or all of the apparatus, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some instances, a degree of Magnetic Resonance Imaging (MRI) compatibility is imparted into the apparatus and/or other elements disclosed herein. For example, the apparatus, and/or components or portions thereof, may be made of a material that does not substantially distort the image and create substantial artifacts (e.g., gaps in the image). Certain ferromagnetic materials, for example, may not be suitable because they may create artifacts in an MRI image. The apparatus, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some instances, the apparatus and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

Having thus described several illustrative examples of the present disclosure, those of skill in the art will readily appreciate that yet other examples may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure's scope is, of course, defined in the language in which the appended claims are expressed.

What is claimed is:

1. A stent comprising:
a tubular body extending from a proximal end to a distal end and defining a lumen therethrough;
a scaffold structure formed from one or more filaments extending between the proximal end and the distal end; and
a polymeric coating disposed along at least a portion of the scaffold structure;
wherein a distal region of the tubular body includes a plurality of openings through the polymeric coating while maintaining the polymeric coating on the one or more filaments.

2. The stent of claim 1, wherein the polymeric coating is configured to increase radial force of the tubular body.

3. The stent of claim 1, wherein the polymeric coating comprises silicone.

4. The stent of claim 1, wherein the distal region extends along about 20% to 40% of a length of the tubular body.

5. The stent of claim 1, wherein the openings of the plurality of openings are spaced about a circumference of the tubular body.

6. The stent of claim 1, wherein the plurality of openings is formed through the polymeric coating after a coating process.

7. The stent of claim 1, wherein the plurality of openings is formed during the polymeric coating process.

8. The stent of claim 1, further comprising at least one radiopaque marker positioned at a transition between the distal region and a fully coated region of the tubular body.

9. The stent of claim 1, further comprising one or more anti-migration elements projecting radially outwardly from the tubular body.

10. The stent of claim 9, wherein the one or more anti-migration elements include proximal anti-migration elements oriented towards the distal end of the stent and distal anti-migration elements oriented towards the proximal end of the stent.

11. The stent of claim 1, further comprising a reinforcing filament interwoven with the scaffold structure.

12. The stent of claim 1, wherein the proximal end includes a proximal cage having an outer diameter greater than an outer diameter of the tubular body.

13. A stent comprising:
a tubular member formed from a single wire extending between a proximal end and a distal end;
a plurality of circumferential segments formed by the single wire;
a polymeric covering extending along at least a portion of the tubular member; and
one or more anti-migration elements formed from the single wire;
wherein a distal region of the tubular member includes a plurality of openings through the polymeric coating while maintaining the polymeric coating on the single wire.

14. The stent of claim 13, wherein the one or more anti-migration elements include proximal anti-migration elements oriented toward the second end and distal anti-migration elements oriented toward the proximal end.

15. The stent of claim 13, further comprising a reinforcing strip extending longitudinally along the tubular member.

16. The stent of claim 13, wherein the polymeric covering increases frictional force between the tubular member and a body lumen.

17. A method of manufacturing a stent, the method comprising:
forming a tubular scaffold structure from one or more filaments;
applying a polymeric coating to the tubular scaffold structure;
forming a plurality of openings through the polymeric coating in a distal region of the tubular scaffold structure while maintaining the polymeric coating on the one or more filaments.

18. The method of claim 17, wherein forming the plurality of openings comprises laser ablation.

19. The method of claim 17, further comprising attaching at least one radiopaque marker at a transition between the distal region and a fully coated region.

20. The method of claim 15, wherein applying the polymeric coating comprises applying silicone.
